(19) United States
(12) Patent Application Publication
(10) Pub. No.: US 2023/0040562 A1
(43) Pub. Date: Feb. 9, 2023

(54) Systems and Methods for Digital Wellness

(71) Applicant: Mirametrix Inc., H3B 1A7 (CA)

(72) Inventors: Michel BOIVIN, Brossard (CA); Jacob FISET, Montreal (CA); Martin GAMSBY, Montreal (CA); Francis GUERIN, Toronto (CA); Simon ST-HILAIRE, Montreal (CA); Cosmin V. LUDUSAN, Montreal (CA)

(21) Appl. No.: 17/970,806

(22) Filed: Oct. 21, 2022

Related U.S. Application Data
(63) Continuation of application No. PCT/CA2021/050550, filed on Apr. 21, 2021.
(60) Provisional application No. 63/013,513, filed on Apr. 21, 2020.

(51) Int. Cl. A61M 21/00

(52) U.S. Cl. CPC A61M 21/00; A61M 2021/005; A61M 2205/50; A61M 2205/3303; A61M 2205/3306

(57) ABSTRACT

Systems and associated methods are provided for monitoring a user while operating a computing device and providing active feedback to said user regarding health and safety best practices associated with operating said computing device. The methods comprise obtaining user biometric data; converting said biometric data into actionable instances of health and safety user device operation use cases; and interacting with the user based on said actionable instances in order to improve or remedy any deviations from recommended health and safety user device operation practices.

### CROSS-REFERENCE TO RELATED APPLICATIONS

The present application is a continuation of International PCT Application No. PCT/CA2021/050550 filed on Apr. 21, 2021, which claims priority from U.S. Provisional Application No. 63/013,513 filed on Apr. 21, 2020, both incorporated herein by reference in their entireties.

### TECHNICAL FIELD

The following relates to systems and methods for promoting digital wellness, particularly to systems and methods for monitoring digital health and safety through user detection and tracking.

### BACKGROUND

At a time when electronic devices, such as work and personal computers, mobile phones and smartwatches, are always on and always connected, people are spending increasing amounts of time using such devices. Smarter, more portable devices paved the way for the novel concept of digital wellness. Digital wellness can be defined from two, somewhat opposing, perspectives.

Digital wellness can be considered to encompass aspects of life where digital devices and applications, through their continuous use, help improve human wellbeing. Some notable examples of such devices and applications include activity trackers, heart rate monitors or smart watches.

However, there is another aspect of digital wellness that is less explored in the digital wellness industry, namely digital hygiene. Digital hygiene may be improved or maintained through use of a collection of devices and applications that discourage or prevent users from exceeding recommended usage time and/or frequency of certain devices, such as work and personal computers, tablets and smartphones.

According to a recent study from the American Academy of Ophthalmology [1], the average office worker spends 1,700 hours per year in front of a computer screen. This extended screen time has led to an increase in complaints of eye strain, dry eyes, headaches and insomnia. According to the same source, eye strain and dry eyes are mainly caused by a reduced blink rate as a consequence of focusing the eyes on computer screens or other digital displays. The blink rate reduction can be anywhere between a third to a half of the average blink rate.

The American Optometric Association lists as common symptoms associated with Digital Eye Strain, also known as Computer Vision Syndrome (CVS): eyestrain, headaches, blurred vision, dry eyes, neck and shoulder pain [2]. Among the main causes behind CVS are improper viewing distances and poor sitting posture.

Poor screen resolution, improper viewing distance and poor sitting posture are identified by the Canadian Centre for Occupational Health and Safety as the main causes behind eye discomfort [3].

While improper digital hygiene can negatively impact physical health, it has been recognized that there may also be social and psychological consequences. According to a 2018 OfficeTeam survey [4], 49% of Canadian workers spend their lunch breaks surfing the web or browsing social media. Yet another survey from 2019, by the Angus Reid Institute [5], indicates that 46% of Canadian parents are concerned that their child is spending too much time in front of a screen.

Many existing technologies that address the problem of digital hygiene are limited to schedule-based screen time applications, software-based blue light filters or simple countdown break reminders. These technologies do not and cannot account for user presence, identity or physical characteristics.

It is desirable to develop improved systems and methods for promoting digital wellness.

### SUMMARY

Provided herein are systems and associated methods for monitoring a user operating a computing device and providing active feedback to said user regarding health and safety best practices associated with operating said computing device. The methods comprise obtaining user biometric data; converting said biometric data into actionable instances of health and safety user device operation use cases; and interacting with the user based on said actionable instances in order to improve or remedy any deviations from recommended health and safety user device operation practices.

In one aspect, provided herein is a system for monitoring digital health and safety of a user operating a computing device, the system comprising:
[a] at least one imaging module configured to acquire image data of a scene including the user;
[b] a user detection and tracking module configured to extract user anthropometric features from the image data;
[c] a biometric data extraction module configured to determine, based on the anthropometric features, biometric data including at least one of a three-dimensional head pose, eye position, eye openness and three-dimensional gaze data; and
[d] a digital wellness monitor module configured to analyze the biometric data to determine at least one digital wellness metric for use in one or more digital wellness methods for providing the user with feedback on their operating of the computing device.

In an implementation, the imaging module includes a visible light sensor, an infrared light sensor, both a dedicated visible light sensor and a dedicated infrared light sensor, a combined visible infrared light sensor, both a visible light sensor and a Time-of-Flight (ToF) sensor, both an infrared light sensor and a TOF sensor, or a combination thereof.

In another implementation, the imaging module is configured to operate in at least one of continuous frame mode, burst frame mode and single frame mode.

In yet another implementation, the biometric data extraction module is further configured to perform one or more of data noise filtering, data fusion and data dimensionality reduction of the biometric data.

In yet another implementation, the digital wellness monitor module is further configured to store the associated digital wellness metrics in one or more databases.

In yet another implementation, the system further comprises a digital wellness assistant module for implementing the one or more digital wellness methods, the digital wellness assistant module interfacing with the digital wellness monitor module in order to provide the user with the feedback, the feedback being one or more of digital wellness alerts, reports, notification messages and prompts.

In yet another implementation, one of the digital wellness methods is a screen time management method comprising:
[a] an initialization step where a screen time counter is started when the user is first detected in the scene as the user looks at a screen;
[b] pausing the screen time counter when the user looks away from the screen;
[c] restarting the screen time counter when the user looks at the screen; and
[d] if a continuously monitored predefined screen time condition is satisfied, repeating the method from step a), otherwise, repeating the method from step b).

In yet another implementation, the predefined screen time condition is that a set screen time limit has been reached by the user within a set time frame.

In yet another implementation, the digital wellness monitor module is communicatively coupled to the local and/or cloud digital wellness logger to store thereon screen time statistics on a daily, weekly, monthly and yearly basis.

In yet another implementation, the digital wellness metrics include screen time since the last screen break, total daily screen time, and screen time over standard or user specified time periods.

In yet another implementation, the digital wellness monitor module is configured to enable an administrator to set the predefined screen time condition.

In yet another implementation, the digital wellness module is further configured to use the set screen time limit to enforce a screen time break by locking and/or turning off the screen for a specified amount of time.

In yet another implementation, the digital wellness methods is an eye care management method.

In yet another implementation, the eye care management method comprises an eye break check to determine whether the user needs to be prompted to take an eye break from the screen and an eye break validation process to confirm that the user has taken the recommended eye break.

In yet another implementation, the eye break is validated when the user is registered by the digital wellness monitor module as looking away from a screen, or screens, for a predefined period of time.

In yet another implementation, the eye break validation follows a rule where every 20 minutes, the user is prompted to look away from the screen at an object 20 feet away for 20 seconds.

In yet another implementation, the digital wellness module is further configured to enable an administrator to enforce the eye care break check and eye break validation.

In yet another implementation, the digital wellness monitor module is further configured to monitor blink rate to estimate digital eye strain and/or eye dryness of the user.

In yet another implementation, the digital wellness monitor module is further configured to prompt the user, via a digital assistant, to take a short screen break, to actively increase their blink rate, or to use eye drops or other dry eye relief products.

In yet another implementation, the digital wellness monitor module is further configured to estimate from the blink rate fatigue or drowsiness of the user.

In yet another implementation, the digital wellness monitor module is configured to take immediate preventative action if the user is determined to have predetermined levels of fatigue or drowsiness and is performing safety critical tasks.

In yet another implementation, the digital wellness monitor module is further configured to implement a body posture monitoring method to determine posture quality of the user.

In yet another implementation, the head pose is a six degrees of freedom head pose.

In yet another implementation, the posture quality is based on whether the user is sitting at a minimum distance from the screen and their head is not at an extreme angle.

In yet another implementation, the digital wellness monitor module is further configured to notify the user, via the digital wellness assistant, to correct body posture, and if the posture quality is determined to be bad, the prompt is persistent until the user moves back beyond the bad posture warning distance.

In yet another implementation, the prompt can be dismissed or snoozed via the digital wellness assistant.

In yet another implementation, the digital wellness monitoring module is further configured to enable an administrator to enforce correction of body posture.

In yet another implementation, the one or more digital wellness methods is screen resolution assistance, and the digital wellness monitoring module is further configured to enable passive or active adjustment of resolution of the screen based on blink rate patterns, eye movements, gaze patterns, head movements, distance of the user from the screen, or a combination thereof.

In yet another implementation, the digital wellness monitoring module is further configured to recommend to the user a change of screen resolution either as a prompt or by changing the resolution to a new resolution and asking the user if they would like to keep the new resolution.

In yet another implementation, the digital wellness monitor module is further configured to change the screen resolution dynamically, without user input, as the user is moving towards or away from the screen.

In yet another implementation, the digital wellness monitoring module is further configured to suggest an improved screen resolution based on historical digital wellness data of the user, the historical digital wellness data including one or more of blink rate behaviour, squinting, and temporal analysis of motion of the user with respect to the display.

In yet another implementation, one of the at least one digital wellness methods is face touching prevention, and the digital wellness monitor module is further configured to monitor face touching by using body tracking of the user, and to and to provide the user with face touching related information.

In yet another implementation, the digital wellness monitor module is configured to enable the user or an administrator to modify the prompts by one or more of setting frequency or time frame thresholds, or enabling snoozing or dismissal of the prompts.

In yet another implementation, the digital wellness monitor module is configured to body tracking information to estimate fatigue, drowsiness and eye strain by monitoring eye rubbing or any other hand and finger contact with the eyes.

In yet another implementation, the digital wellness monitor module is configured to detect onychophagia, and to prompt the user with a warning or a series of escalating warnings which can ultimately direct the user to take a screen break.

In another aspect, provided is a method for monitoring digital health and safety of a user operating a computing device, the method comprising:
[a] acquiring, using an imaging module, image data of a scene including the user;
[b] extracting, using a user detection and tracking module, user anthropometric features from the image data;
[c] using a biometric data extraction module, determining based on the anthropometric features, biometric data including at least one of a three-dimensional head pose, eye position, eye openness and three-dimensional gaze data; and
[d] analyzing using a digital wellness monitoring module the biometric data to determine at least one digital wellness metric for use in one or more digital wellness methods for providing the user with feedback on their operating of the computing device.

In an implementation of the method, the imaging module acquires the image data using a visible light sensor, an infrared light sensor, both a dedicated visible light sensor and a dedicated infrared light sensor, a combined visible infrared light sensor, both a visible light sensor and a Time-of-Flight (ToF) sensor, both an infrared light sensor and a TOF sensor, or a combination thereof.

In another implementation of the method, the imaging module is operated in at least one of continuous frame mode, burst frame mode and single frame mode.

In yet another implementation, the method further comprises performing one or more of data noise filtering, data fusion and data dimensionality reduction of the biometric data before step d).

In yet another implementation, the method further comprises storing the associated digital wellness metrics in one or more databases.

In yet another implementation, the method further comprises providing, via a digital wellness assistant module interfacing with the digital wellness monitor module, the user with the feedback, the feedback being one or more of digital wellness alerts, reports, notification messages and prompts.

In yet another implementation, one of the digital wellness methods is a screen time management method comprising:
[a] an initialization step where a screen time counter is started when the user is first detected in the scene as the user looks at a screen;
[b] pausing the screen time counter when the user looks away from the screen;
[c] restarting the screen time counter when the user looks at the screen; and
[d] if a continuously monitored predefined screen time condition is satisfied, repeating the method from step a), otherwise, repeating the method from step b).

In yet another implementation, the predefined screen time condition is that a set screen time limit has been reached by the user within a set time frame.

In yet another implementation, the method further comprises storing on one or more databases screen time statistics on a daily, weekly, monthly and yearly basis.

In yet another implementation, the digital wellness metrics include screen time since the last screen break, total daily screen time, and screen time over standard or user specified time periods.

In yet another implementation, the digital wellness monitor module is used to enable an administrator to set the predefined screen time condition.

In yet another implementation, the digital wellness monitor module uses the set screen time limit to enforce a screen time break by locking and/or turning off the screen for a specified amount of time.

In yet another implementation, one of the digital wellness methods is an eye care management method.

In yet another implementation, the eye care management method comprises an eye break check to determine whether the user needs to be prompted to take an eye break from the screen and an eye break validation process to confirm that the user has taken the recommended eye break.

In yet another implementation, the eye break is validated when the user is registered by the digital wellness monitor module as looking away from a screen, or screens, for a predefined period of time.

In yet another implementation, the eye break validation follows a rule where every 20 minutes, the user is prompted to look at an object 20 feet away for 20 seconds.

The method of claim 49, wherein the digital wellness module is further configured to enable an administrator to enforce the eye care break check and eye break validation.

In yet another implementation, the digital wellness monitor module is used to monitor blink rate to estimate digital eye strain and/or eye dryness of the user.

In yet another implementation, the digital wellness monitor module is used to prompt the user, via a digital assistant, to take a short screen break, to actively increase their blink rate, or to use eye drops or other dry eye relief products.

In yet another implementation, the digital wellness monitor module is used to estimate from the blink rate fatigue or drowsiness of the user.

In yet another implementation, the digital wellness monitor module is configured to take immediate preventative action if the user is determined to have predetermined levels of fatigue or drowsiness and is performing safety critical tasks.

In yet another implementation, the digital wellness monitor module is further configured to implement a body posture monitoring method to determine posture quality of the user.

In yet another implementation, the head pose is a six degrees of freedom head pose.

In yet another implementation, the posture quality is based on whether the user is sitting at a minimum distance from the screen and their head is not at an extreme angle.

In yet another implementation, the digital wellness monitor module is further configured to notify the user, via a digital wellness assistant, to correct body posture, and if the posture quality is determined to be bad, the prompt is persistent until the user moves back beyond the bad posture warning distance.

In yet another implementation, the prompt can be dismissed or snoozed via the digital wellness assistant.

In yet another implementation, the digital wellness monitoring module is further configured to enable an administrator to enforce correction of body posture.

In yet another implementation, the one or more digital wellness methods is screen resolution assistance, and the digital wellness monitoring module is further configured to enable passive or active adjustment of resolution of the screen based on blink rate patterns, eye movements, gaze patterns, head movements, distance of the user from the screen, or a combination thereof.

In yet another implementation, the digital wellness monitoring module is further configured to recommend to the user a change of screen resolution either as a prompt or by changing the resolution to a new resolution and asking the user if they would like to keep the new resolution.

In yet another implementation, the digital wellness monitor module is further configured to change the screen resolution dynamically, without user input, as the user is moving towards or away from the screen.

In yet another implementation, the digital wellness monitoring module is further configured to suggest an improved screen resolution based on historical digital wellness data of the user, the historical digital wellness data including one or more of blink rate behaviour, squinting, and temporal analysis of motion of the user with respect to the display.

In yet another implementation, one of the at least one digital wellness methods is face touching prevention, and the digital wellness monitor module is further configured to monitor face touching by using body tracking of the user, and to and to provide the user with face touching related information.

In yet another implementation, the digital wellness monitor module is configured to enable the user or an administrator to modify the prompts by one or more of setting frequency or time frame thresholds or enabling snoozing or dismissal of the prompts.

In yet another implementation, the digital wellness monitor module is configured to body tracking information to estimate fatigue, drowsiness and eye strain by monitoring eye rubbing or any other hand and finger contact with the eyes.

In yet another implementation, the digital wellness monitor module is configured to detect onychophagia, and to prompt the user with a warning or a series of escalating warnings which can ultimately direct the user to take a screen break.

In yet another aspect, provided is a computing device comprising one or more processors and at least one storage medium, the at least one storage medium containing non-transitory computer-readable instructions for execution by the one or more processors to cause the one or more processors to perform any of the methods.

### BRIEF DESCRIPTION OF THE DRAWINGS

Embodiments will now be described by way of example only with reference to the appended drawings wherein:

FIG. 1 is a block diagram of a system for monitoring the digital wellness of a user.

FIGS. 2a and 2b are flow charts illustrating examples of computer executable instructions performed by the system when providing a screen time counter and/or screen time manager digital wellness function.

FIG. 3 is an example illustration of a communication interface through which the digital wellness assistant shown in FIG. 1 can present screen time counter analytics to the user.

FIG. 4 is a flow chart illustrating an example set of computer executable instructions performed by the system when providing an eye break digital wellness function.

FIG. 5 is a block diagram of an example of the interaction steps between the user and the digital wellness assistant when an eye break digital wellness functionality is provided.

FIG. 6 is a flow chart illustrating an example set of computer executable instructions performed by the system when providing a bad posture monitoring digital wellness function.

FIG. 7 is a block diagram of an example of interaction steps between the user and the digital wellness assistant when a bad posture monitoring digital wellness functionality is provided.

FIG. 8 is a flow chart illustrating an example of computer executable instructions performed by the system when providing an optimal display resolution digital wellness function.

### DETAILED DESCRIPTION

Provided herein are systems and methods for user-aware digital wellness use cases.

It will be appreciated that for simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It will also be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

It has been recognized by numerous official health and safety guidelines that people who perform intensive computer work for prolonged periods of time may have an increased risk of developing a number of specific health problems. Such guidelines recommend that employees take regular short screen time breaks and maintain a correct sitting posture and screen viewing distance. While the problems, the associated negative effects and mitigation strategies are clearly laid out in health and safety guides, the implementation rests solely on the user.

It has also been recognized that the mitigation strategies may only be as effective as the user’s ability to remember to blink more frequently when viewing a computer screen for prolonged periods of time, to sit properly or to maintain the recommended distance from the screen. Keeping in mind these strategies while performing intensive computer work tends to be difficult for the computer user.

Enabling a user to have a passive role in mitigation strategy implementation and enforcement may reduce the user’s cognitive load and assist them in focusing on their tasks, which may in turn increase effectiveness of mitigation strategies.

Provided are systems and methods for implementing digital wellness strategies, or use cases, by means of detecting and tracking at least one aspect of the computer user’s body, face, eye or gaze.

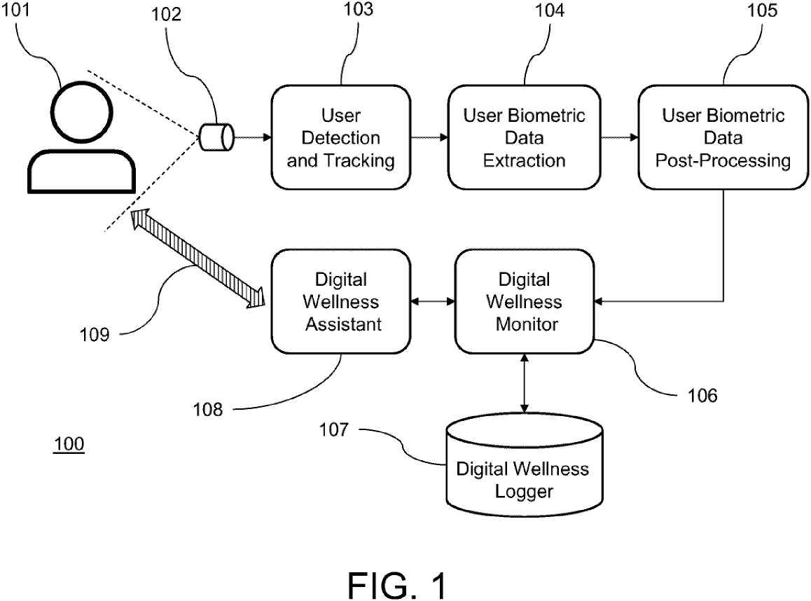

Turning to FIG. 1, illustrated is a system for digital wellness 100 comprising an imaging module 102 configured to provide an image feed of a scene containing at least one aspect of a computer user’s body, face, eye or gaze. The system 100 further comprises a user detection and tracking unit 103 for receiving the image feed from the imaging module 102. The user detection and tracking unit 103 may be configured to run a collection of machine executable algorithms for detecting and tracking one or more of the user’s body, face, eye and gaze. Following the detection and tracking of the user’s body, face, eye or gaze, the user detection and tracking unit 103 may output a set of user features. The set of user features may include, but is not limited to, face contour landmarks, mouth and nose landmarks, eye contour landmarks, pupil and iris landmarks. The user biometric data extraction unit 104 may be configured to process the set of user features in order to extract user-specific characteristics. The set of user-specific characteristics may include, but is not limited to, six degrees of freedom (6DoF) head pose information, eye position, eye openness, three-dimensional gaze data, and user identity. The unit 104 may be configured to send the extracted user biometric data to a biometric data post-processing unit 105. The biometric data post-processing unit 105 may be configured to carry out data processing methods including, but not limited to, data noise filtering, data fusion, data dimensionality reduction. The system 100 further comprises a digital wellness monitor 106 configured to receive post-preprocessed biometric data from the post-processing unit 105. The digital wellness monitor 106 can use the post-processed biometric data to compute a series of digital wellness metrics and enable implementation of associated digital wellness use cases. The wellness monitor 106 may also be responsible for storing the digital wellness metrics into a local and/or cloud digital wellness logger 107. The digital wellness monitor 106 is also connected to the digital wellness assistant 108 which serves as a user communication interface 109 via which the digital wellness alerts, reports and any other type of messages are presented to the user 101.

The imaging module 102 may include a visible light sensor, an infrared light sensor, both a dedicated visible light sensor and a dedicated infrared light sensor, a combined visible-infrared light sensor, both a visible light sensor and a Time-of-Flight (ToF) sensor, both an infrared light sensor and a TOF sensor, one or more other appropriate sensors, or any combination thereof.

The imaging module 102 may be configured to operate in one ore more modes including, but not limited to, continuous frame mode, burst frame mode and single frame mode.

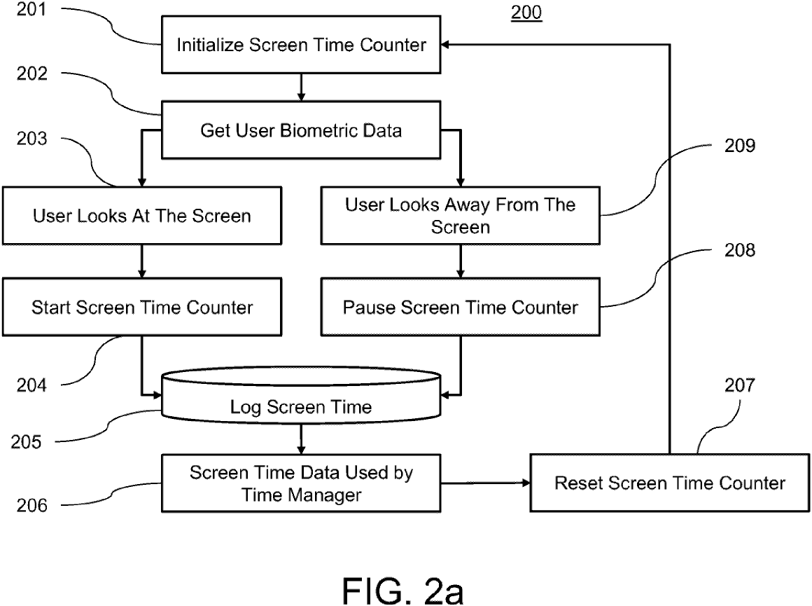

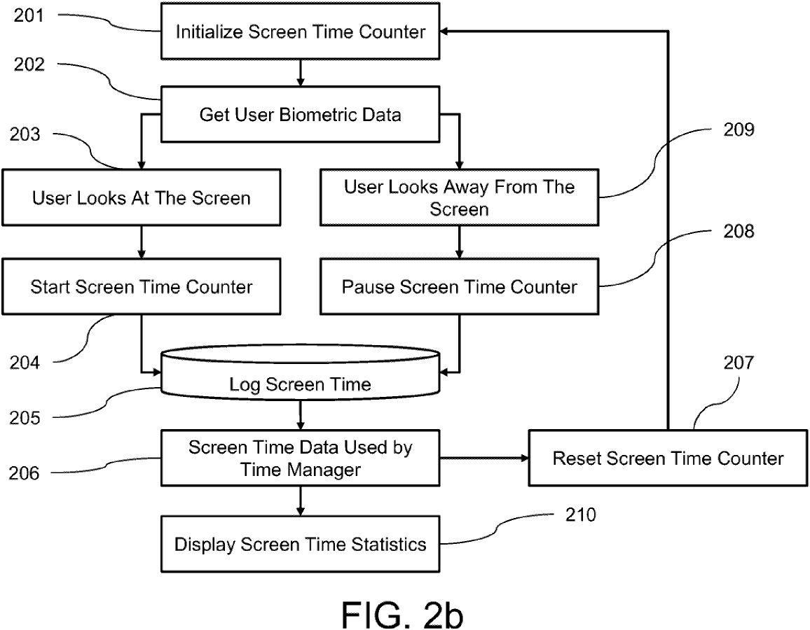

In one aspect, the digital wellness monitor 106 can measure a user’s screen time using a screen time counter loop 200 (FIG. 2). The loop 200 may be referred to hereinafter as a “screen time counter use case”. The screen time counter loop 200 can begin with an initialization step 201 which, in turn, activates a user biometric data logic 202. The user biometric data logic 202 may provide a user state loop for starting and pausing the screen time counter depending on whether the user is looking at the screen. The screen time counter may be enabled when the user looks at the screen (203,204). The screen time counter may be paused (208) if the user is detected as looking elsewhere (209). Most or all starting and pausing events may be logged at step 205 to be used by a screen time manager block at step 206. At step 206, the screen time manager, which may be part of the digital wellness monitor 106, may produce or initiate screen time counter reset commands at step 207 for local events and reinitialize the screen time counter (201), thereby restarting the loop 200. Such local events may include, for example, the user 101 reaching a predefined screen time limit.

In an example embodiment of the screen time counter use case, the screen time manager logic 206, part of the digital wellness monitor 106, is also connected to the global digital wellness logger 107 which can store screen time statistics on a daily, weekly, monthly and yearly basis. The stored screen time statistics may be retrieved at the user’s 101 or system administrator’s request for display and analysis via the digital wellness assistant 108 and the user communication interface 109.

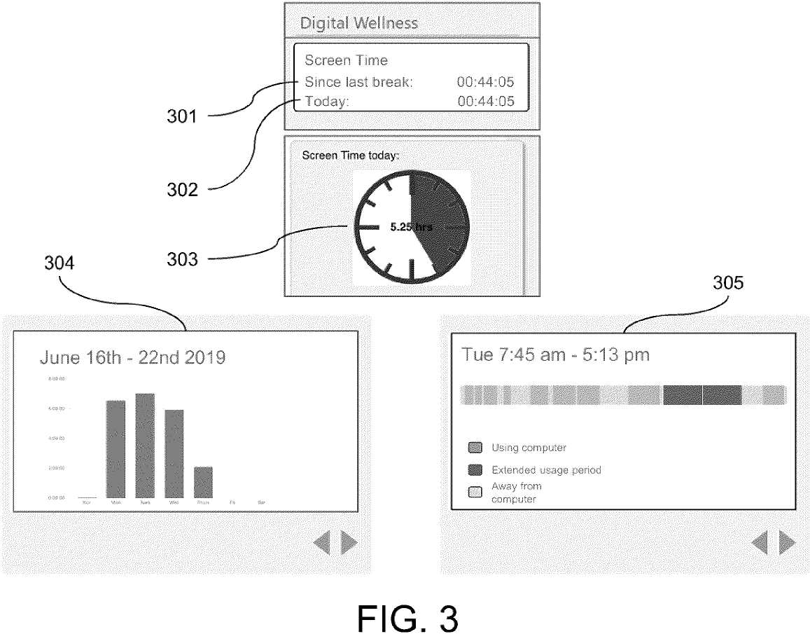

Turning to FIG. 3, the digital wellness assistant 108, via the user communication interface 109, can display to the user screen time analytics, such as the screen time since the last screen break 301, the total daily screen time, in a numerical format 302 or a graphical format 303. The screen time analytics can be also expanded to standardized or user specified time periods 304 or time of day standard or user specified periods 305.

Other screen time counter functionality may include, inter alia, more advanced screen time manager 206 options such as maximum screen time limits or screen time privileges. These functions can be set by an administrator on the local machine or via a remote connection and can be customized for different users, if the device is shared. The screen time limits may be used by the screen time manager 206 to enable a mandatory screen time break by locking and or turning off the display for a specified amount of time.

In one embodiment, the screen time manager functionality of the digital wellness monitor 106 can require only 6DoF head pose user data to be enabled. In another embodiment, the digital wellness monitor 106 can utilize the user’s gaze to enable the screen time manager functionality. A yet another embodiment, the digital wellness monitor 106 can make use of the user’s identity to allow for multiple screen time manager user profiles on shared devices.

In yet another embodiment, the screen time manager in the digital wellness monitor 106 can be set up to work with multiple displays, where the user biometric data from the unit 105, such as 6DoF head pose or gaze, can be used to monitor all active displays and account for the cumulative screen time, shared between the active displays.

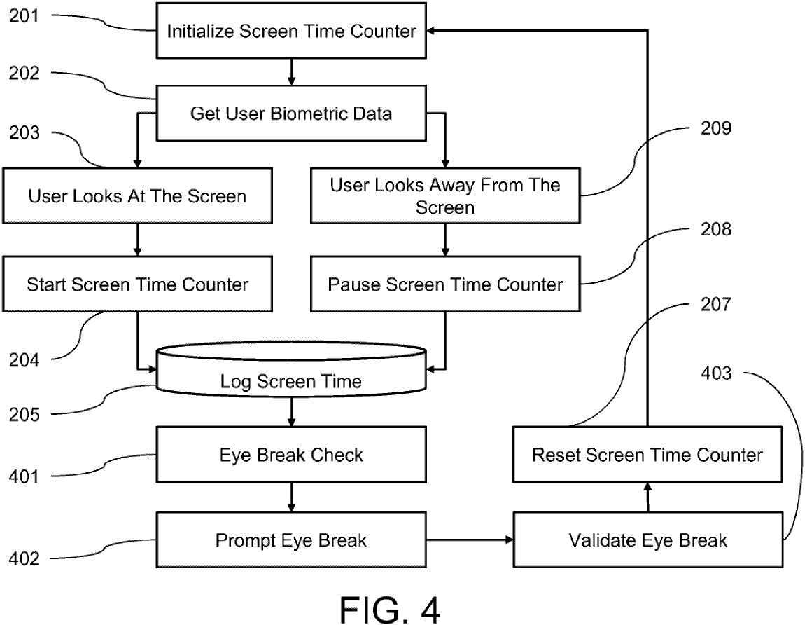

The digital wellness monitor 106 can be configured to include an eye care manager to implement an eye care management method, alone or in combination with the screen time manager 206 methods. In one instance, the eye care management method includes an eye break reminder and validation process. As shown in FIG. 4 the logical process flow can be the same as the screen time counter loop 200, but with the addition of an eye break check 401 performed based on the screen time logged at step 205. The eye break check, or process 401 can determine whether the user 101 needs to be prompted to take an eye break from the screen.

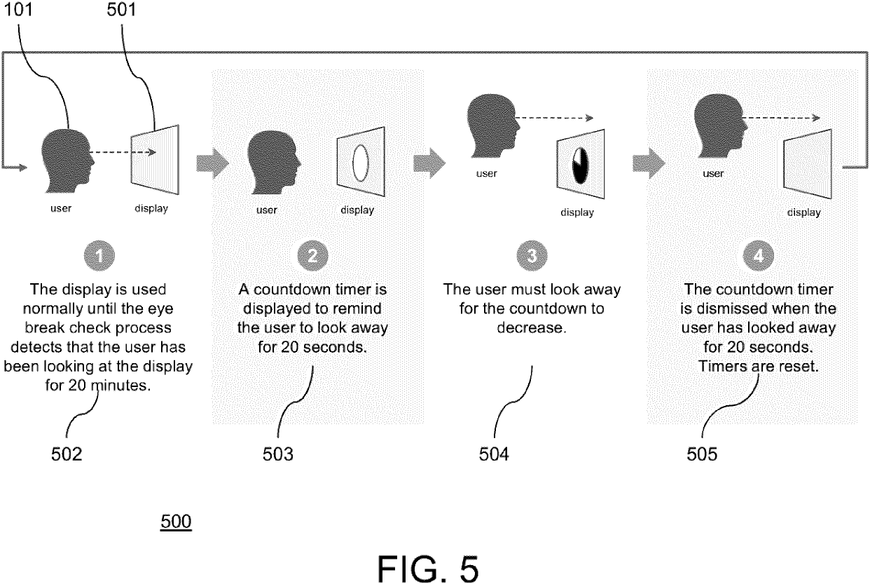

In one instance, and with reference to FIG. 5, the eye break check 401 follows the “20-20-20” rule, as outlined in [1] - “every 20 minutes, look at something 20 feet away for 20 seconds”. The 20-20-20 rule is a method recommended by various ophthalmology and optometry organizations as a way to prevent digital eye strain. If the 20 minutes of continuous screen time have passed 502, the eye break check 401 will notify the user 101 via the eye break prompt 402. The eye break prompt 402 may be carried out by the digital wellness assistant 108 and can interact with the user 101 via the user communication interface 109. The process 402 instructs the user 101 to look away from the computer display, or displays, for a predefined period of time 503. After the predefined period of time for which the user 101 is instructed to look into the distance, away from the display, the eye break is validated 403, to ensure that the recommended eye break has taken place. The validation process 403 uses user biometric data, such as, for example, gaze and head pose to confirm that the user 101 is not looking at the display during the eye break activation. The eye break counter only counts down from the predefined time when the user 101 is not looking at the display. Any interruption caused by the user looking back at the display 504 may pause the eye break countdown timer, which can resume when the user again looks away from the display 505.

In one embodiment, the eye break validation 403 changes the display by, for example, blurring the display’s contents, dimming the display or turning the display off for the duration of the predefined eye break.

In one embodiment, the eye break check can be dismissed by the user or snoozed for a set period of time. The eye break intervals and duration can also be changed from the default “20-20-20” rule.

In yet another embodiment, the eye break settings, such as the check and the duration can be set by a system administrator as mandatory digital wellness features. When this is done, the user biometric data post-processing 105 can include anti-spoofing checks to ensure that the digital wellness functionality is not circumvented by the user.

In another embodiment, the display or displays are shared by multiple users, the user biometric data extraction 104 includes user identification which allows the digital wellness monitor 106 to distinguish between multiple users and function accordingly.

Another embodiment of the digital wellness monitor 106 includes as part of the eye care functionality, alone or in combination with other use cases, a blink rate monitor to mitigate digital eye strain and dry eyes. The blink rate can obtained through user eye feature extraction (with unit 104), post-processing (with unit 105) and subsequent analysis by the digital wellness monitor 106. Based on this analysis, the digital wellness monitor 106 can prompt the user 101 via the digital assistant 108 to mitigate the reduced blink rate problem. By way of example, the user can be instructed to take a short screen break, to actively increase their blink rate, or to use eye drops or other dry eye relief products in order to reduce digital eye strain.

In another embodiment, the blink rate can also logged in the digital wellness logger 107 for user-specific analytics and daily, weekly, monthly and yearly report retrieval.

In yet another embodiment, the blink rate functionality can also used to estimate user fatigue and drowsiness. In such embodiment, the digital wellness monitor can be configured to quickly or immediately take preventative action if the user is performing safety critical tasks.

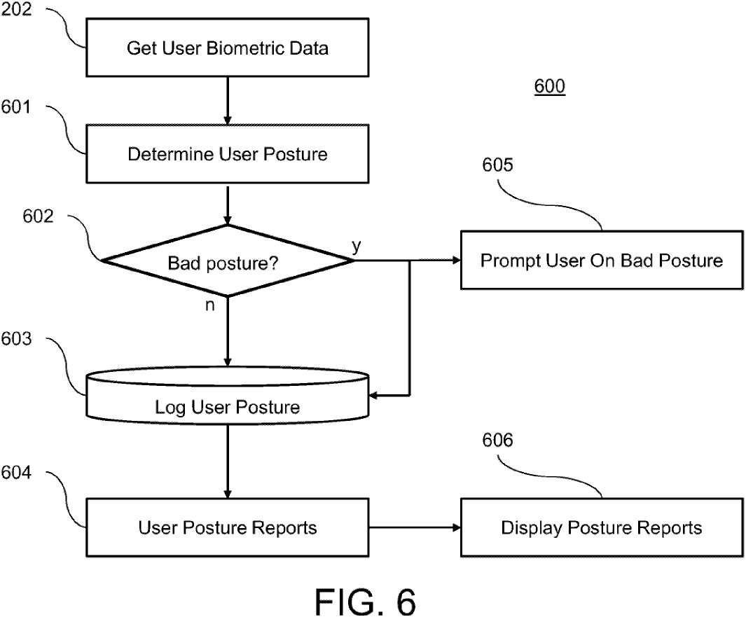

Another embodiment of the digital wellness monitor 106 can implement, either as a standalone feature or together with other use cases, a body posture monitoring method. An example embodiment of a body posture monitoring method 600 is shown in FIG. 6. The method 600 is enabled by using the user’s 6DoF head pose as an indicator of good or bad posture. The user biometric data, obtained at step 202, is used to determine the user’s posture at step 601 by analyzing head pose and distance from the display. It is determined at step 602 whether the posture is good or bad based on whether the user is sitting at a minimum distance from the display and their head is not at an extreme angle. This indicates that the user may be hunching over the keyboard, experiencing neck pain and added spinal pressure. If bad posture is detected at step 602, the digital wellness monitor will prompt the user (step 605) and log user posture behavior for later reporting (step 604) and later display (step 606).

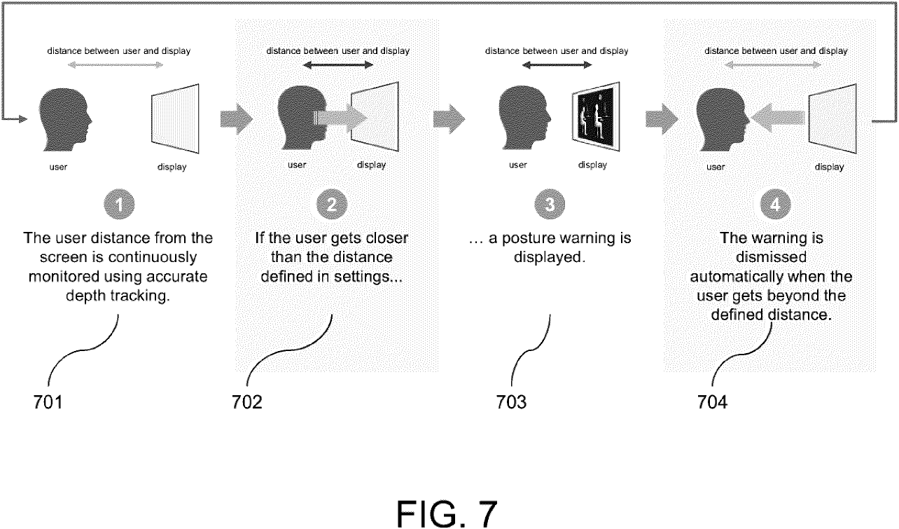

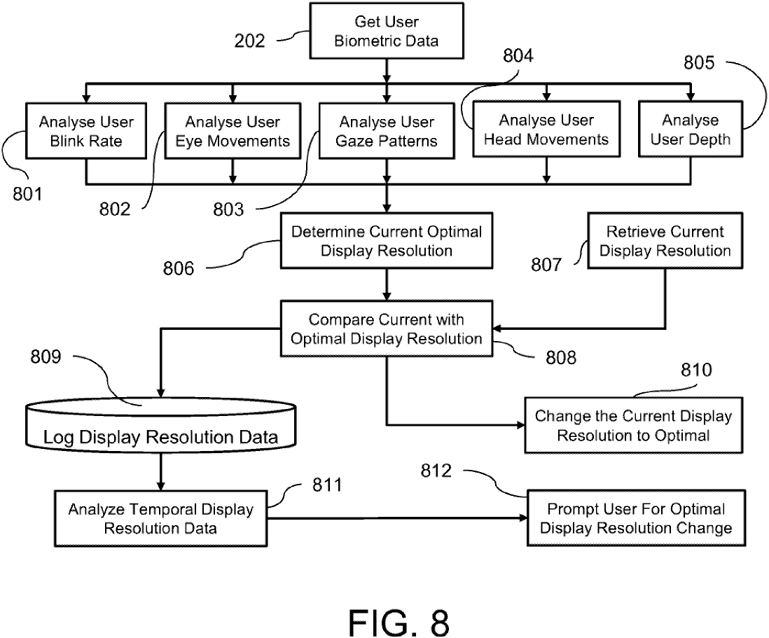

Turning to FIG. 7, in yet another embodiment of the user posture monitoring, the digital wellness monitor can analyze the user’s distance from the display 701 prompt the user 703 if they get closer than a predefined distance from said display. In one embodiment, the bad posture prompt 704 is persistent until the user moves back beyond the bad posture warning distance 704.

The posture monitoring and the bad posture warning can be dismissed or snoozed via the digital wellness assistant 108. The posture monitoring setting can also be restricted by an administrator, making the defined behavior and corrective bad posture action mandatory.

Yet another embodiment of the digital wellness monitor 106 includes, either as a standalone feature or together with other use cases, as part of the eye care functionality an optimal screen resolution assistant. With the use of the user biometric data, the digital wellness monitor can either actively or passively change the display resolution to an optimal one based on multiple user metrics. By way of example, the digital wellness monitor can determine the optimal screen resolution from the available ones, based on, inter alia, blink rate patterns 801, eye movements 802, gaze patterns 803, head movements 804, the user’s distance from the display 805, or any combination thereof. In this embodiment, the imaging system 102 is found in the same spatial plane as the display. The digital wellness assistant 108 can then recommend the user a change of display resolution either as a prompt 812 or by actually changing the resolution 810 and asking the user if they would like to keep the new resolution.

In another embodiment, the digital wellness assistant 108 changes the display resolution dynamically 810, without user input, as the user is moving towards or away from the display. The digital wellness assistant may thus ensure that the user is provided most of the time, preferably always, with an appropriate or optimal available resolution where display readability is balanced with digital eye care. An added advantage of this embodiment can be identified in the area of portable devices, where the dynamic resolution control can also help with battery consumption.

In yet another embodiment, the optimal resolution can be suggested by the digital wellness assistant 108 based on the user’s historical digital wellness data 809, such as blink rate behaviour, squinting, and/or temporal analysis of the user’s motion with respect to the display.

In yet another embodiment, the digital wellness monitor is configured to utilize body tracking alone or in combination with head positioning to enable body posture monitoring.

In yet another embodiment, the digital wellness monitor 106 can use body tracking to monitor face touching and prompt the user with warning about potential health hazards associated with viral and bacterial transmission via face touching. The functionality can be customized by the user, by setting frequency or time frame thresholds, snoozing or dismissing it altogether.

In yet another embodiment, the digital wellness monitor 106 can use body tracking to monitor fatigue, drowsiness and eye strain by monitoring eye rubbing or any other hand and finger contact with the eyes.

In yet another embodiment, the digital wellness monitor 106 can be configured to include onychophagia mitigation (i.e. nail biting) and prevention functionality. The digital wellness assistant 108 can prompt the user with a warning or a series of escalating warnings which can ultimately direct the user to take a screen break.

It will be appreciated that any module or component exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the systems and/or devices described herein, any component of or related thereto, or accessible or connectable thereto. Any application or module herein described may be implemented using computer readable/executable instructions that may be stored or otherwise held by such computer readable media.

Although the above principles have been described with reference to certain specific examples, various modifications thereof will be apparent to those skilled in the art as outlined in the appended claims.

### REFERENCES

American Academy of Ophthalmology, Protect Your Eyes From Too Much Screen Time, Mar. 11, 2019, https://www.aao.org/newsroom/news-releases/detail/protect-your-eyes-from-too-much-screen-time.

American Optometric Association, Computer Vision Syndrome, 2020, https://www.aoa.org/patients-and-public/caring-for-your-vision/protecting-your-vision/computer-vision-syndrome.

Canadian Centre for Occupational Health and Safety, Eye Discomfort in the Office, Apr. 8, 2020, https://www.ccohs.ca/oshanswers/ergonomics/office/eye discomfort.html.

OfficeTeam Surveys, Canadian Workers Prioritize Screen Time Over Face Time at Lunch, September 2018, https://www.roberthalf.ca/en/canadian-workers-prioritize-screen-time-over-face-time-at-lunch.

[5] Angus Reid Institute, Digital Dopamine: Half of Canadian parents concerned their child spends too much time on their devices, Sep. 3, 2019, http://angusreid.org/screen-time-kids/.

1. A system for monitoring digital health and safety of a user operating a computing device, the system comprising:
at least one imaging module configured to acquire image data of a scene including the user;
a user detection and tracking module configured to extract user anthropometric features from the image data;
a biometric data extraction module configured to determine, based on the anthropometric features, biometric data including at least one of a three-dimensional head pose, eye position, eye openness and three-dimensional gaze data; and
a digital wellness monitor module configured to analyze the biometric data to determine at least one digital wellness metric for use in one or more digital wellness methods for providing the user with feedback on their operating of the computing device.

2. The system of claim 1, wherein the imaging module is configured to operate in at least one of continuous frame mode, burst frame mode and single frame mode and includes a visible light sensor, an infrared light sensor, both a dedicated visible light sensor and a dedicated infrared light sensor, a combined visible infrared light sensor, both a visible light sensor and a Time-of-Flight (ToF) sensor, both an infrared light sensor and a TOF sensor, or a combination thereof.

3. The system of claim 1, wherein the biometric data extraction module is further configured to perform one or more of data noise filtering, data fusion and data dimensionality reduction of the biometric data.

4. The system of claim 1, further comprising a digital wellness assistant module for implementing the one or more digital wellness methods, the digital wellness assistant module interfacing with the digital wellness monitor module in order to provide the user with the feedback, the feedback being one or more of digital wellness alerts, reports, notification messages and prompts.

5. The system of claim 1, wherein one of the digital wellness methods is a screen time management method comprising:
a) an initialization step where a screen time counter is started when the user is first detected in the scene as the user looks at a screen;
b) pausing the screen time counter when the user looks away from the screen;
c) restarting the screen time counter when the user looks at the screen; and
d) if a continuously monitored predefined screen time condition is satisfied, repeating the method from step a), otherwise, repeating the method from step b).

6. The system of claim 5, wherein the screen time management method further comprises one or more of:
a predefined screen time condition that a set screen time limit has been reached by the user within a set time frame;
a set of digital wellness metrics comprising at least one of screen time since the last screen break, total daily screen time, and screen time over standard or user specified time periods;
configuring the digital wellness monitor module to enable an administrator to set the predefined screen time condition; and
configuring the digital wellness module to use the set screen time limit to enforce a screen time break by locking and/or turning off the screen for a specified amount of time.

7. The system of claim 6, wherein the digital wellness monitor module is communicatively coupled to the local and/or cloud digital wellness logger to store thereon screen time statistics on a daily, weekly, monthly and yearly basis.

8. The system of claim 1, wherein one of the digital wellness methods is an eye care management method comprising one or more of:
an eye break check to determine whether the user needs to be prompted to take an eye break from the screen and an eye break validation process to confirm that the user has taken the recommended eye break;
validating the eye break when the user is registered by the digital wellness monitor module as looking away from a screen, or screens, for a predefined period of time;
prompting the user every 20 minutes to look away from the screen at an object 20 feet away for 20 seconds; and
further configuring the digital wellness module to enable an administrator to enforce the eye care break check and eye break validation.

9. The system of claim 1, wherein the digital wellness monitor module is further configured to monitor blink rate to estimate digital eye strain and/or eye dryness of the user by means of a method comprising one or more of:
prompting the user, via a digital assistant, to take a short screen break, to actively increase their blink rate, or to use eye drops or other dry eye relief products;
estimating from the blink rate fatigue or drowsiness of the user; and
taking immediate preventative action if the user is determined to have predetermined levels of fatigue or drowsiness and is performing safety critical tasks.

10. The system of claim 1, wherein the digital wellness monitor module is further configured to implement a body posture monitoring method to determine posture quality of the user, comprising:
an initial six degrees of freedom head pose estimation step;
determining the posture quality is based on whether the user is sitting at a minimum distance from the screen and their head is not at an extreme angle;
notifying the user, via the digital wellness assistant, to correct body posture, and if the posture quality is lower than a predefined posture quality value, the prompt is persistent until the posture quality is at or above the predefine posture quality value;
providing the user with the option to dismiss or snooze the prompt via the digital wellness assistant; and
further configuring the digital wellness monitoring module to enable an administrator to enforce correction of body posture.

11. The system of claim 1, wherein the one or more digital wellness methods is screen resolution assistance, and the digital wellness monitoring module is further configured to enable passive or active adjustment of resolution of the screen based on blink rate patterns, eye movements, gaze patterns, head movements, distance of the user from the screen, or a combination thereof.

12. The system of claim 11, wherein the digital wellness monitoring module is further configured to:
recommend to the user a change of screen resolution as a prompt;
change the screen resolution to a new screen resolution and asking the user if they would like to keep the new screen resolution;
to change the screen resolution dynamically, without user input, as the user is moving towards or away from the screen; or
to suggest an improved screen resolution based on historical digital wellness data of the user, the historical digital wellness data including one or more of blink rate behaviour, squinting, and temporal analysis of motion of the user with respect to the display.

13. The system of claim 1, wherein one of the at least one digital wellness methods is face touching prevention, and the digital wellness monitor module is further configured to:
monitor face touching by using body tracking of the user, and to and to provide the user with face touching related information; and
enable the user or an administrator to modify the prompts by one or more of setting frequency or time frame thresholds, or enabling snoozing or dismissal of the prompts.

14. The system of claim 1, wherein the digital wellness monitor module is configured to acquire body tracking information to estimate fatigue, drowsiness and eye strain by monitoring eye rubbing or any other hand and finger contact with the eyes.

15. The system of claim 1, wherein the digital wellness monitor module is configured to detect onychophagia, and to prompt the user with a warning or a series of escalating warnings which can ultimately direct the user to take a screen break.

16. A computer implemented method for monitoring digital health and safety of a user operating a computing device, the method comprising:
with at least one imaging module, acquiring image data of a scene including the user;
with a user detection and tracking module extracting user anthropometric features from the image data;
with a biometric data extraction module, determining based on the anthropometric features, biometric data comprising at least one of a three-dimensional head pose, eye position, eye openness and three-dimensional gaze data; and
with a digital wellness monitor module, analyzing the biometric data to determine at least one digital wellness metric for use in one or more digital wellness methods for providing the user with feedback on their operating of the computing device.

17. The method of claim 16, wherein the imaging module operates in at least one of continuous frame mode, burst frame mode and single frame mode and includes a visible light sensor, an infrared light sensor, both a dedicated visible light sensor and a dedicated infrared light sensor, a combined visible infrared light sensor, both a visible light sensor and a Time-of-Flight (ToF) sensor, both an infrared light sensor and a TOF sensor, or a combination thereof.

18. The method of claim 16, further comprising performing with the biometric data extraction module, one or more of data noise filtering, data fusion and data dimensionality reduction of the biometric data.

19. The method of claim 16, further comprising implementing the one or more digital wellness methods with a digital wellness assistant module, the digital wellness assistant module interfacing with the digital wellness monitor module in order to provide the user with the feedback, the feedback being one or more of digital wellness alerts, reports, notification messages and prompts.

20. The method of claim 16, wherein one of the digital wellness methods is a screen time management method comprising:
a) an initialization step where a screen time counter is started when the user is first detected in the scene as the user looks at a screen;
b) pausing the screen time counter when the user looks away from the screen;
c) restarting the screen time counter when the user looks at the screen; and
d) if a continuously monitored predefined screen time condition is satisfied, repeating the method from step a), otherwise, repeating the method from step b).

21. The method of claim 16, wherein the screen time management method further comprises one or more of:
implementing a predefined screen time condition that a set screen time limit has been reached by the user within a set time frame;
monitoring a set of digital wellness metrics comprising at least one of screen time since the last screen break, total daily screen time, and screen time over standard or user specified time periods;
enabling an administrator to set the predefined screen time condition; and
when the set screen time limit is reached, enforcing a screen time break by locking and/or turning off the screen for a specified amount of time.

22. The method of claim 21, wherein the digital wellness monitor module is communicatively coupled to the local and/or cloud digital wellness logger and the method further comprises storing thereon screen time statistics on a daily, weekly, monthly and yearly basis.

23. The method of claim 16, wherein one of the digital wellness methods is an eye care management method comprising one or more of:
performing an eye break check to determine whether the user needs to be prompted to take an eye break from the screen and an eye break validation process to confirm that the user has taken the recommended eye break;
validating the eye break when the user is registered by the digital wellness monitor module as looking away from a screen, or screens, for a predefined period of time;
prompting the user every 20 minutes to look away from the screen at an object 20 feet away for 20 seconds; and
enabling an administrator to enforce the eye care break check and eye break validation.

24. The method of claim 16, wherein the digital wellness monitor module is further configured to monitor blink rate to estimate digital eye strain and/or eye dryness of the user by means of a method comprising one or more of:
prompting the user, via a digital assistant, to take a short screen break, to actively increase their blink rate, or to use eye drops or other dry eye relief products;
estimating from the blink rate fatigue or drowsiness of the user; and
taking immediate preventative action if the user is determined to have predetermined levels of fatigue or drowsiness and is performing safety critical tasks.

25. The method of claim 16, wherein the digital wellness monitor module is further configured to implement a body posture monitoring method to determine posture quality of the user, the body posture monitoring method comprising:
an initial six degrees of freedom head pose estimation step;
determining the posture quality is based on whether the user is sitting at a minimum distance from the screen and their head is not at an extreme angle;
notifying the user, via the digital wellness assistant, to correct body posture, and if the posture quality is lower than a predefined posture quality value, the prompt is persistent until the posture quality is at or above the predefine posture quality value;
providing the user with the option to dismiss or snooze the prompt via the digital wellness assistant; and
further configuring the digital wellness monitoring module to enable an administrator to enforce correction of body posture.

26. The method of claim 16, wherein the one or more digital wellness methods is screen resolution assistance, and the digital wellness monitoring module enables passive or active adjustment of resolution of the screen based on blink rate patterns, eye movements, gaze patterns, head movements, distance of the user from the screen, or a combination thereof.

27. The method of claim 26, further comprising:
recommending to the user a change of screen resolution as a prompt;
changing the screen resolution to a new screen resolution and asking the user if they would like to keep the new screen resolution;
changing the screen resolution dynamically, without user input, as the user is moving towards or away from the screen; or
suggesting an improved screen resolution based on historical digital wellness data of the user, the historical digital wellness data including one or more of blink rate behaviour, squinting, and temporal analysis of motion of the user with respect to the display.

28. The method of claim 16, wherein the at least one digital wellness methods comprises face touching prevention comprising the steps of:
monitoring face touching by using body tracking of the user, and to and to provide the user with face touching related information; and
enabling the user or an administrator to modify the prompts by one or more of setting frequency or time frame thresholds, or enabling snoozing or dismissal of the prompts.

29. The method of claim 16, further comprising, with the digital wellness monitor module, acquiring body tracking information to estimate fatigue, drowsiness and eye strain by monitoring eye rubbing or any other hand and finger contact with the eyes.

30. The method of claim 16, wherein the digital wellness monitor module is configured to detect onychophagia, the method further comprising:
if onychophagia is detected, prompting the user with a warning or a series of escalating warnings which can ultimately direct the user to take a screen break.

31. A computing device comprising one or more processors and at least one storage medium, the at least one storage medium containing non-transitory computer-readable instructions for execution by the one or more processors to cause the one or more processors to perform the method of claim 16.
